(19) United States
(12) Patent Application Publication
(10) Pub. No.: US 2012/0135053 A1
(43) Pub. Date: May 31, 2012

(54) NANOPARTICULATE TELMISARTAN COMPOSITIONS AND PROCESS FOR THE PREPARATION THEREOF

(76) Inventors: Genovéva Filipcsei, Budapest (HU); Zsolt Ötvös, Csongrad (HU); Katalin Pongrácz, Budapest (HU); Ferenc Darvas, Budapest (HU)

(21) Appl. No.: 13/379,151

(22) PCT Filed: Jun. 18, 2010
(86) PCT No.: PCT/HU2010/000070

§ 371 Date: Feb. 13, 2012

(30) Foreign Application Priority Data
Jun. 19, 2009 (HU) P0900383
Apr. 19, 2010 (HU) P1000215

(51) Int. Cl. A61K 31/4184; A61P 9/12; A61K 9/14; B82Y 5/00

(52) U.S. Cl. 424/400; 514/394; 977/773; 977/788

(57) ABSTRACT

The present invention is directed to nanostructured (nanoparticulated) Telmisartan compositions, process for the preparation thereof and pharmaceutical compositions containing them. The nanoparticles of Telmisartan according to the invention have an average particle size of less than about 600 nm. Telmisartan is an angiotensin II receptor antagonist (ARB) used in the management of hypertension.

### FIELD OF THE INVENTION

The present invention is directed to nanostructured (nanoparticulated) Telmisartan compositions, process for the preparation thereof and pharmaceutical compositions containing them.

The nanoparticles of Telmisartan according to the invention have an average particle size of less than about 600 nm. Telmisartan is an angiotensin II receptor antagonist (ARB) used in the management of hypertension.

### BACKGROUND OF THE INVENTION

A. Background Regarding to Nanoparticle Formation/Production

Nanoparticles development for Pharmaceutical Applications deals with emerging new technologies for developing customized solutions for drug delivery systems. The drug delivery systems should positively impact the rate of absorption, distribution, metabolism, and excretion of the drug or other related chemical substances in the body. In addition, the drug delivery system should allow the drug to bind to its target receptor and influence that receptor's signaling and activity. Drug delivery materials should be compatible, easy to bind with a particular drug, and able to degrade into fragments after use that are either metabolized or driven out via normal excretory routes.

A different approach is to produce the active ingredient (API) in nanoparticulate form.

Telmisartan composition is described in US 20020094997, US2009/0030057 A1, US 20090012140 and EP 1797872 A1 patent applications.

The API nanoparticles can be made using, for example, milling, homogenization, precipitation techniques, or supercritical fluid techniques, as is known in the art. Methods of making nanoparticulate compositions are also described in U.S. Pat. No. 5,718,388, U.S. Pat. No. 5,862,999, U.S. Pat. No. 5,665,331, U.S. Pat. No. 5,543,133, U.S. Pat. No. 5,534,270.

B. Background Regarding Telmisartan

Telmisartan is chemically described as 4′-[(1,4′-dimethyl-2′-propyl [2,6′-bi-1H-benzimidazol]-1′-yl)methyl]-[1,1′-biphenyl]-2-carboxylic acid. Its empirical formula is C₃₃H₃₀N₄O₂, its molecular weight is 514.63, and its structural formula is:

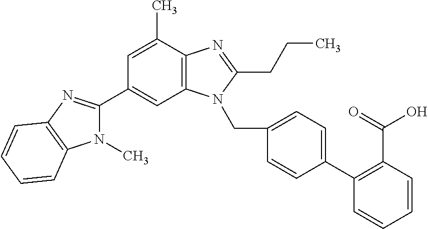

Telmisartan is a white to slightly yellowish solid. It is practically insoluble in water and in the pH range of 3 to 9, sparingly soluble in strong acid (except insoluble in hydrochloric acid), and soluble in strong base.

Telmisartan is available as tablets for oral administration, containing 20 mg, 40 mg or 80 mg of Telmisartan. The tablets contain the following inactive ingredients: sodium hydroxide, meglumine, povidone, sorbitol, and magnesium stearate. The tablets are hygroscopic and require protection from moisture.

### Pharmacological Properties

Following oral administration, peak concentrations (Cmax) of Telmisartan are reached in 0.5-1 hour after dosing. Food slightly reduces the bioavailability of Telmisartan, with a reduction in the area under the plasma concentration-time curve (AUC) of about 6% with the 40 mg tablet and about 20% after a 160 mg dose. The absolute bioavailability of Telmisartan is dose dependent. At 40 and 160 mg the bioavailability was 42% and 58%, respectively. The pharmacokinetics of orally administered Telmisartan is nonlinear over the dose range 20-160 mg, with greater than proportional increases of plasma concentrations (Cmax and AUC) with increasing doses. Telmisartan shows bi-exponential decay kinetics with a terminal elimination half life of approximately 24 hours. Plasma concentrations of Telmisartan with once daily dosing are about 10-25% of peak plasma concentrations. Telmisartan has an accumulation index in plasma of 1.5 to 2.0 upon repeated once daily dosing.

### Metabolism and Elimination

Following, either intravenous or oral administration of ¹⁴C-labeled Telmisartan, most of the administered dose (>97%) was eliminated unchanged in feces via biliary excretion; only minute amounts were found in the urine (0.91% and 0.49% of total radioactivity, respectively).

Telmisartan is metabolized by conjugation to form a pharmacologically inactive acylglucuronide; the glucuronide of the parent compound is the only metabolite that has been identified in human plasma and urine. After a single dose, the glucuronide represents approximately 11% of the measured radioactivity in plasma. The cytochrome P450 isoenzymes are not involved in the metabolism of Telmisartan.

Total plasma clearance of Telmisartan is >800 mL/min. Terminal half-life and total clearance appears to be independent of dose.

### Distribution

Telmisartan is highly bound to plasma proteins (>99.5%), mainly albumin and α1—acid glycoprotein. Plasma protein binding is constant over the concentration range achieved with recommended doses. The volume of distribution for Telmisartan is approximately 500 liters indicating additional tissue binding.

### Side Effects

The most frequently spontaneously reported side effects include: headache, dizziness, asthenia, coughing, nausea, fatigue, weakness, edema, face edema, lower limb edema, angioneurotic edema, urticaria, hypersensitivity, sweating increased, erythema, chest pain, atrial fibrillation, congestive heart failure, myocardial infarction, blood pressure increased, hypertension aggravated, hypotension (including postural hypotension), hyperkalemia, syncope, dyspepsia, diarrhea, pain, urinary tract infection, erectile dysfunction, back pain, abdominal pain, muscle cramps (including leg cramps), myalgia, bradycardia, eosinophilia, thrombocytopenia, uric acid increased, abnormal hepatic function/liver disorder, renal impairment including acute renal failure, anemia, and increased CPK.

Because of the insolubility of Telmisartan in water, there is a need in the art to enhance the lipophilicity/bioavailability/increase the absorption/reduce the side effect/decrease the dosage/reduce the food effect in order to overcome the problems associated with the use of prior conventional Telmisartan formulations. Moreover, these problems can be solved by surface modification to decrease the first pass effect or modify the metabolism of Telmisartan. Beside the traditional formulation of Telmisartan, the transdermal application could decrease the time which is needed to reach the desired effect of Telmisartan. The present invention satisfies this need.

### DESCRIPTION OF THE INVENTION

The present invention describes the nanostructured (nanoparticulated) Telmisartan composition with enhanced lipophilicity/bioavailability/increased absorption and dissolution rate/reduced side effect/decreased dosage.

As exemplified in the examples below, not every combination of stabilizer will result in a stable nanoparticle formation. It was discovered, that stable, Telmisartan nanoparticles can be made by continuous flow method, preferably by microfluidic based continuous flow method, using selected stabilizers

The invention comprises nanostructured Telmisartan having an average particle size of less than about 600 nm.

The nanostructured Telmisartan according to the invention has an average particle size between 600 nm and 50 nm, preferably 200 nm and 50 nm.

Further aspect of the invention is a stable nanostructured Telmisartan composition comprising:
(a) nanostructured Telmisartan having an average particle size of less than about 600 nm; and
(b) at least stabilizer.

The composition of the invention is prepared in a continuous flow reactor, preferably in a microfluidic based continuous flow reactor.

In the composition of the invention the average particle size of Telmisartan is preferably between 600 nm and 50 nm, preferably 200 nm and 50 nm.

In the composition of the invention: (a) the Telmisartan is present in an amount selected from the group consisting of from about 99.5% to about 0.001%, from about 95% to about 0.1%, and from about 90% to about 0.5%, by weight, based on the total combined weight of the Telmisartan and at least one stabilizer, not including other excipients; (b) the stabilizer is present in an amount selected from the group consisting of from about 0.5% to about 99.999% by weight, from about 5.0% to about 99.9% by weight, and from about 10% to about 99.5% by weight, based on the total combined dry weight of the Telmisartan and at least one stabilizer, not including other excipients; or (c) a combination of (a) and (b).

In the composition of the invention the Telmisartan can be used in a crystalline phase, an amorphous phase, a semi-crystalline phase, a semi-amorphous phase, and co-crystal, and in mixtures thereof in any polymorph form.

For the preparation of the composition of the invention stabilizers include nonionic, anionic, cationic, ionic polymers/surfactants and zwitterionic surfactants can be used. Combinations of more than one stabilizer can also be used in the invention. Useful stabilizers which can be employed in the invention include, but are not limited to, known organic and inorganic pharmaceutical excipients. Such excipients include various polymers, low molecular weight oligomers, natural products, and surfactants.

Representative examples of stabilizers include hydroxypropyl methylcellulose, hydroxypropylcellulose, poly(vinylpyrrolidone), sodium lauryl sulfate, gelatin, dextran, stearic acid, glycerol monostearate, cetostearyl alcohol, sorbitan esters, polyoxyethylene castor oil derivatives, polyoxyethylene sorbitan fatty acid esters (e.g., the commercially available Tween® products such as e.g., Tween® 20 and Tween® 80 (ICI Speciality Chemicals); polyethylene glycols (e.g., Carbowax® 3550 and 934 (Union Carbide), poly(meth)acrylate-based polymers and copolymers (Eudargit®), acetic acid ethenyl ester polymer with 1-ethenyl-2-pyrrolidinone (PVP/VA copolymers), sodium dodecyl benzene sulfonate, tocopheryl polyethylene glycol succinates, polyethoxylated castor oils and its derivatives, polyoxyethylene stearates, methylcellulose, hydroxyethylcellulose, cellulose acetate phthalate, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics, which are block copolymers of ethylene oxide and propylene oxide); poloxamines (e.g., Tetronic, also known as Poloxamine, which is a tetrafunctional block copolymer derived from sequential addition of propylene oxide and ethylene oxide to ethylenediamine (BASF Wyandotte Corporation, Parsippany, N.J.); PEG-phospholipid, PEG-cholesterol, PEG-cholesterol derivative, PEG-vitamin A, PEG-vitamin E, lysozyme, poly(2-ethyl-2-oxazoline), poly(methyl vinyl ether), random copolymers of vinyl pyrrolidone and vinyl acetate, such as Plasdone S630, and the like.

Examples of useful ionic stabilizers include, but are not limited to polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), benzalkonium chloride, hexadecyltrimethylammonium bromide, hexyldesyltrimethylammonium bromide (HDMAB), and poly(vinylpyrrolidone)-2-dimethylaminoethyl methacrylate dimethyl sulfate.

Advantages of the composition of the invention include, but are not limited to: (1) smaller tablet or other solid dosage form size and beneficial transdermal/topical application; (2) lower doses of drug required to obtain the same pharmacological effect as compared to conventional forms of Telmisartan; (3) increased bioavailability as compared to conventional forms of Telmisartan; (4) improved pharmacokinetic profiles; (5) an increased rate of dissolution for Telmisartan nanoparticles as compared to conventional forms of the same active compound; (6) modified metabolism of Telmisartan nanoparticles.

For the preparation of the composition of the invention methods can be used comprising a continuous solvent-antisolvent precipitation using one or more stabilizers or a continuous chemical precipitation using one or more stabilizers to form nanoparticles without Telmisartan form conversion or amorphous drug formation and without pre-sterilization.

Another aspect of the invention is a process for the preparation of nanostructured Telmisartan, comprising precipitating nanostructured Telmisartan from an appropriate solution of Telmisartan comprising one or more stabilizers if desired in the presence of a pharmaceutically acceptable acid in a continuous flow reactor.

As a continuous flow reactor a microfluidic based continuous flow reactor may be used.

The microfluidics based continuous flow reactor used is described in the publication Microfluid Nanofluid DOI 10.1007/s10404-008-0257-9 by I. Homyak, B. Borcsek and F. Darvas.

Preferably the process may be carried out by (1) dissolving Telmisartan and optionally one or more stabilizers in a suitable solvent; (2) adding the formulation from step (1) to a solution comprising optionally stabilizer(s) if desired in the presence of a pharmaceutically acceptable acid; and (3) precipitating the formulation from step (2).

An other preferred embodiment of the process is where the process is carried out by (1) dissolving Telmisartan and optionally one or more stabilizers in a suitable solvent; (2) adding the formulation from step (1) to a solution comprising one or more stabilizers in desired in the presence of a pharmaceutically acceptable acid; and (3) precipitating the formulation from step (2).

Most preferably the process of the invention is carried out by (1) dissolving Telmisartan and one or more stabilizers in an alkali-hydroxide solution; (2) adding the formulation from step (1) to a solution of a pharmaceutically acceptable acid comprising optionally one or more stabilizers; and (3) precipitating the formulation from step (2).

As solvents (a) two different solvents miscible with each other may be used, where Telmisartan is soluble only in one of them, or (b) the same solvent may be used in the two steps, where the polyelectrolyte complex of Telmisartan forms nanostructured particles, practically, with the restriction that the applied stabilizer is soluble in the solvents used.

Such solvents may be alkali-hydroxide solutions, preferably sodium-hydroxide solution, dimethyl-sulfloxide, ethanol, i-propanol, tetrahydrofuran, acetone, methyl-ethyl-ketone, dimethyl-formamide, diethylene-glycol-ethyl-ether preferably.

As pharmaceutically acceptable acids acetic acid, citric acid, maleic acid, oxalic acid, formic acid, benzoic acid, and the like may be used.

The particle size of the nanoparticulate Telmisartan can be influenced by the solvents used, the flow rate and the Telmisartan—stabilizer ratio.

Another aspect of the invention is directed to the good/instantaneous redispersibility of solid nanosized form of Telmisartan in biologically relevant mediums, e.g.; physiological saline solution, pH=2.5 HCl solution.

Another aspect of the invention is a pharmaceutical composition comprising a stable nanoparticulate Telmisartan or composition of it according to the invention and optionally pharmaceutically acceptable auxiliary materials.

The pharmaceutical composition of the invention can be formulated: (a) for administration selected from the group consisting of oral, pulmonary, rectal, colonic, parenteral, intracistemal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The compositions can be formulated by adding different types of excipients for oral administration in solid, liquid, vaginal, rectal, local (powders, ointments or drops), or topical administration, and the like.

A preferred dosage form of the invention is a solid or liquid (cream/ointment) dosage form, although any pharmaceutically acceptable dosage form can be utilized.

For oral delivery into the human body nanoparticles can be also administered as their aqueous dispersion as the final dosage form. This is a way of delivery without further processing after nanoparticle formation. However, poor stability of the drug or polymer in an aqueous environment or poor taste of the drug may require the incorporation of the colloidal particles into solid dosage forms, i.e. into capsules and tablets.

Alternatively, the aqueous dispersion of the colloidal particles can be incorporated into the solid dosage form as a liquid, for example by granulation of suitable fillers with the colloidal dispersion to form a granulation. Such granules can subsequently be filled into capsules or be compressed into tablets. Alternatively, through layering of the dispersion onto e.g. sugar-pellets as carriers in a fluidized bed a solid form for nanoparticles can be. These ways of manufacturing tablet cores, or granules or pellets can potentially by followed by a coating step to reveal a film-coated tablet or film coated granules in a capsule as the final dosage form.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alginates, gelatin, poly(vinylpyrrolidone), sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the Telmisartan, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

The pharmaceutical compositions of the invention show enhanced lipophilicity/bioavailability/increased absorption and dissolution rate/reduced side effect/faster onset of action, so they can be used in a decreased dosage as compared to conventional Telmisartan form in the treatment of hypertension.

The present invention is also directed to methods for management of hypertension Telmisartan nanoparticles disclosed herein.

### A. Preferred Characteristics of the Telmisartan Nanoparticles of the Invention

### 1. Increased Bioavailability

The nanoparticulate Telmisartan compositions of the invention are proposed to exhibit increased bioavailability, faster onset of action, reduced food effect and require smaller doses as compared to prior known, conventional Telmisartan formulations.

### Example 1

### In Vivo Pharmacokinetic Tests Male Sprague-Dawley Rats in Fasted Condition: Comparison of Reference Active Pharmaceutical Ingredient, Marketed Pritor Tablet and Nanostructured Telmisartan

### Experimental Protocols

### Comparative In Vivo Pharmacokinetic Tests in Male Sprague-Dawley Rats in Fasted Condition

The single oral dose of reference Telmisartan was 30 mg/kg, and that of nanostructured Telmisartan formulation of example 8 was 223.8 mg/kg which corresponds to 30 mg/kg active agent. Both test substances were administered via gastric tube in a dosing volume of 5 ml/kg. The vehicle of the test items was sterile 0.9% NaCl solution and the suspension was kept homogenous by continuous stirring during treatment in order to minimize the error resulting from the sedimentation.

Comparative In Vivo Pharmacokinetic Tests in Male Sprague-Dawley Rats in Fed Condition Administering pH=5 Nanostructured and Pritor Suspension

The single oral dose of reference Telmisartan was 30 mg/kg, and that of nanostructured Telmisartan formulation of example 8 was 223.8 mg/kg which corresponds to 30 mg/kg active agent. Both test substances were administered via gastric tube in a dosing volume of 5 ml/kg. The vehicle of the test items was sterile 0.9% NaCl solution and it pH was adjusted to pH=5 by 1 N HCl solution. The suspension was kept homogenous by continuous stirring during treatment in order to minimize the error resulting from the sedimentation.

### Animals

Male Wistar rats (purchased from Laboratory Animal Center, University of Szeged) were maintained on a standard pellet rodent diet (Bioplan Ltd, Isaszeg, Hungary) under temperature and light-controlled conditions with tap water available ad libitum. The acclimatization period was at least 4 days. Rats were randomized into groups of 6 and each group was used for blood sampling at different time period after Telmisartan treatment. All animals were fasting for 16 hours before oral treatment. Animals were anesthetized with halothane and blood has been withdrawn by cardiac puncture 15, 30, 45, 60, 120 and 360 minutes after Telmisartan treatment. Water was available immediately after treatment for all animals. Rats in the last group (sacrificed at 360 min) had access to standard rodent food 120 minutes after the treatment. Serum samples were prepared by centrifugation (7000 rpm, 10 min, 4° C.) of the clotted blood within 60 minutes and were stored at −20° C. till analysis.

### Sample Preparation

An aliquot of 200 μl serum was combined with 20 μl of internal standard working solution and 1.2 ml acetonitrile for protein precipitation. The mixture was vortexed for 1 min and centrifuged at 12000 rpm for 10 min at 4° C. The supernatants were evaporated to dryness under a stream of nitrogen at 40° C. and reconstituted with 200 μl of water—methanol (50:50 v/v) and 20 μl was injected into the HPLC system.

### Statistical Analysis

Unpaired t-test was used to statistically compare the serum concentrations belonging to the same time points. Statistical analysis and graph drawing were carried out be GraphPad Prism 4.0 (GraphPad Software, San Diego, USA).

### Results

a) Comparison of Reference and Nanostructured Telmisartan

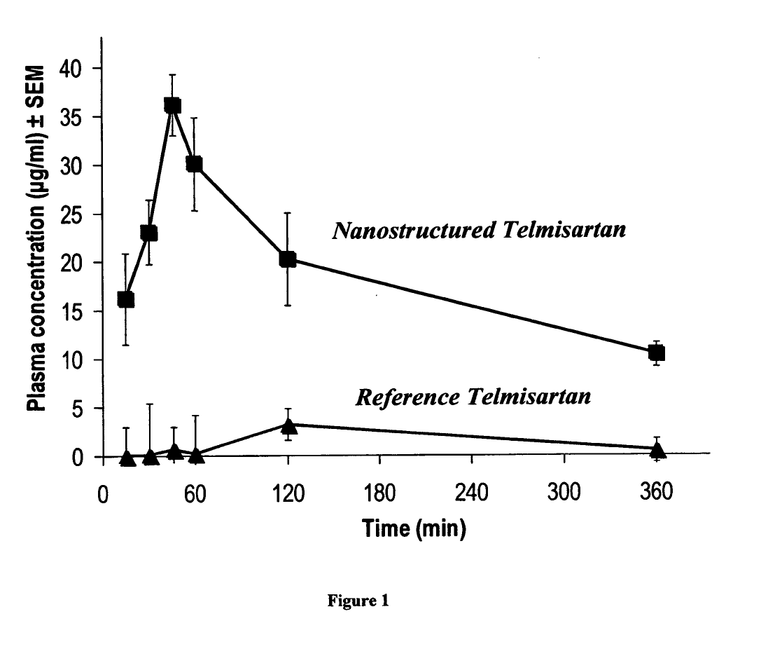

Both reference active pharmaceutical and nanostructured Telmisartan treatment resulted in a detectable serum concentration exhibiting a biphasic profile in the 15-360 min interval after the oral administration of 30 mg/kg test substance. The absorption of Telmisartan from nanostructured formula is obviously faster and more complete than after the administration of reference substance. Following nanostructured Telmisartan treatment the maximal serum concentration (Cₘₐₓ) was determined at 45 min, while reference preparation resulted in Cₘₐₓ at 120 min (FIG. 1).

Area under the serum concentration curve between 15 and 360 min (AUC_{15-360 min}) has been calculated to characterize the extent of the absorption of the test items. Nanostructured Telmisartan resulted in an AUC_{15-360 min} value of 6412 μg·min/ml while this value after reference treatment was 940.1 μg·min/ml. The ratio of the two AUC values, (AUC_{15-360 min (nanosized)}/AUC_{15-360 min (reference)}) was 6.82.

FIG. 1.: Serum concentrations of Telmisartan after oral administration of 30 mg/kg nanostructured and reference test substance

b) Comparison of Marketed Pritor Tablet and Nanostructured Telmisartan

Both reference active pharmaceutical and nanostructured Telmisartan treatment resulted in a detectable serum concentration exhibiting a biphasic profile in the 15-360 min interval after the oral administration of 30 mg/kg test substance. No statistically different serum concentrations were found between the two treatments corresponding to the same time (unpaired t-test). Following nanostructured Telmisartan treatment the maximal serum concentration (Cₘₐₓ) was determined at 45 min, while Pritor 40 mg tablet resulted in Cₘₐₓ at 60 min.

Area under the serum concentration curve between 15 and 360 min (AUC_{15-360 min}) has been calculated to characterize the extent of the absorption of the test items. Nanostructured Telmisartan resulted in an AUC_{15-360 min} value of 6412 μg·min/ml while this value after Pritor 40 mg tablet treatment was 8069 μg·min/ml. The ratio of the two AUC values (AUC_{15-360 min (nanostructured)}/AUC_{15-360 min (Pritor 40 mg tablet)}) was 0.795.

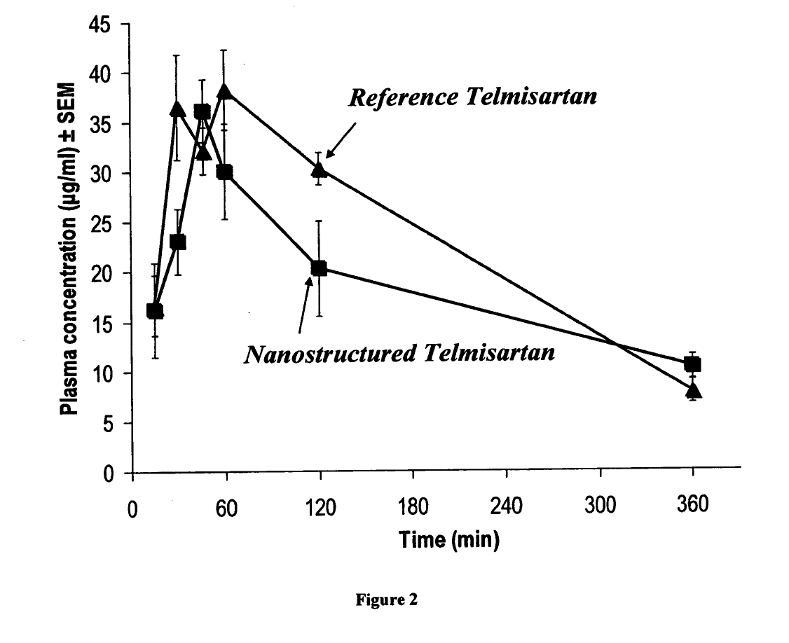

Serum concentration of Telmisartan after 30 min of administration exhibits a minimum. However, comparison of concentrations at 15, 30 and 45 min (ANOVA followed by Newman-Keuls posthoc test) revealed no statistical difference. Overall, the presented results clearly indicate that the absorption of nanostructured Telmisartan is statistically identical with that obtained after the administration of a commercially available drug preparation (Pritor 40 mg tablet) (FIG. 2).

FIG. 2.: Serum concentrations of Telmisartan after oral administration of 30 mg/kg nanostructured and Pritor test substance

c) Elimination of the Effect of Naoh Presence

To evaluate the effect of sodium hydroxide on solubility, PK test was performed administering Pritor tablet and nanostructured Telmisartan of example 8 in physiological saline solution which was adjusted to pH=5. The absorption of Telmisartan was followed under fed condition.

Both reference active pharmaceutical and nanostructured Telmisartan treatment resulted in a detectable serum concentration exhibiting a biphasic profile in the 15-360 min interval after the oral administration of 30 mg/kg test substance in fed condition. The absorption of Telmisartan from nanostructured formula is obviously faster and more complete than after the administration of reference substance. Following nanostructured Telmisartan treatment the maximal serum concentration (Cₘₐₓ) was determined at 30 min, while reference preparation resulted in Cₘₐₓ at 120 min.

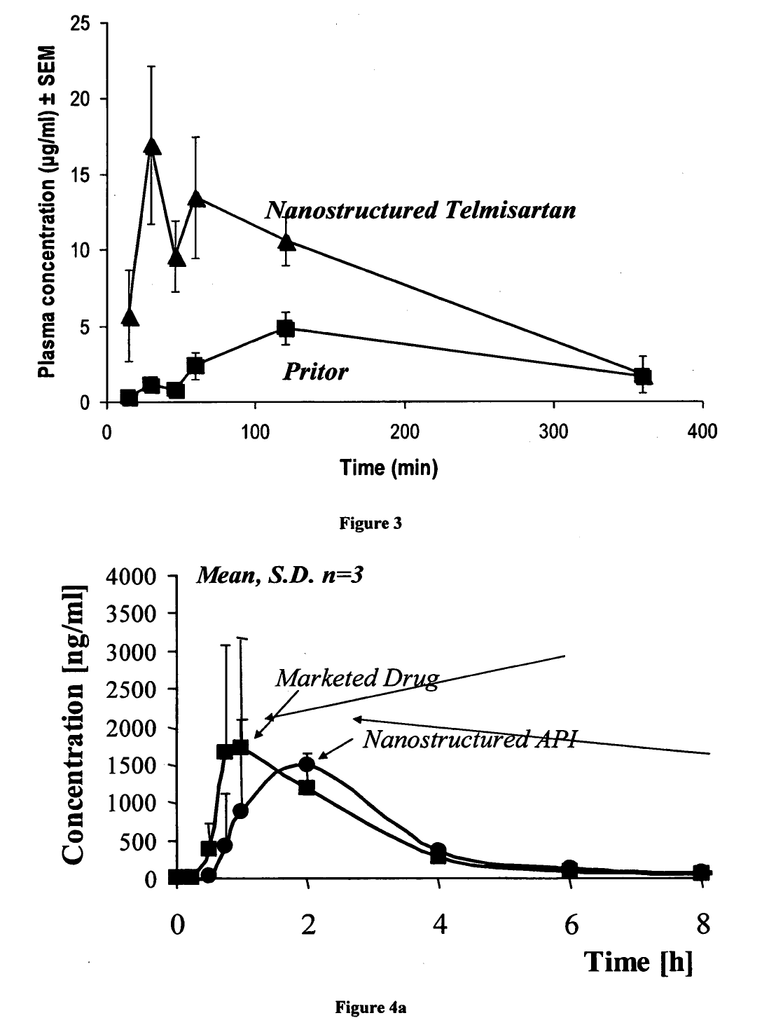

Area under the serum concentration curve between 15 and 360 min (AUC_{15-360 min}) has been calculated to characterize the extent of the absorption of the test items. Nanostructured Telmisartan resulted in an AUC_{15-360 min} value of 2744 μg·min/ml while this value after reference treatment was 1242 μg·min/ml. The ratio of the two AUC values (AUC_{15-360 min (nanosized)}/AUC_{15-360 min (reference)}) was 2.21 (FIG. 3).

FIG. 3.: Serum concentrations of Telmisartan after oral administration of 30 mg/kg nanostructured and Pritor test substance at pH=5 under fed condition

Table 1.: Results of pharmacokinetic tests in rats

### Example 2

### Comparative In Vivo Pharmacokinetic Test on Female Beagle Dogs in Fed/Fasted Condition

This study was designed to compare the pharmacokinetic parameters obtained after the oral administration of different Telmisartan formulations in fed and fasted animals. The following formulations were used:
Test formulation: nanostructured Telmisartan formulation of example 8
Test formulation: nanostructured Telmisartan formulation of example 8 and NaOH measured into wafer capsule for administration
Reference formulation: commercially available Pritor 40 mg tablet (administered in wafer capsule) manufactured by Pfizer AG.

### Experimental Protocols

Comparative in vivo pharmacokinetic test was a cross-over, single dose, two period study. Three female Beagle dogs received a single oral dose of the test and the reference formulations containing the same amount of Telmisartan. The dose of the active ingredient was 40 mg/animal. The plasma concentrations of Telmisartan were quantified using a reliable bioanalytical method.

To characterize the systemic exposure of Telmisartan the main pharmacokinetic parameters (Cₘₐₓ, Tₘₐₓ, and AUC) were determined for the individual plasma level versus time curves. The parameters obtained after administration of the test formulation were compared to those obtained for the reference tablet.

### Animals

The Beagle dog is suitable non-rodent species for pharmacokinetic studies and is acceptable to regulatory authorities. The dog is readily available, easy to handle, house and dose and suitable for investigation of the whole plasma level curve in each individual animal. The systemic exposure was investigated in six dogs.

The study was conducted according to the Guide for the Care and Use of Laboratory Animals, NRC, 1996 and in compliance with the principles of the Hungarian Act 1998:XXVIII. regulating animal protection.

### Food and Feeding

The animals received ssniff Hd-H diet for dogs produced by Ssniff, Spezialdiaten GmbH. The food was offered daily 300 g/dog approximately at the same time. The next morning the remaining food was taken away. Before the administrations, the animals were fasted overnight (at least 12 h). On the treatment day, animals randomized in the fasted group received the food approx. 4 hours after the administration. Animals randomized into the fed groups received approximately 150 g standard diet. The other 150 g food was offered at approximately 4 hours after the administration.

### Blood Collection and Plasma Separation

For determination of plasma levels of Telmisartan approximately 3 ml of blood was collected in plastic vials with lithium heparin as anticoagulant. The time points of blood collection were the followings in both periods: pre-dose (0 min), 15 min, 30 min, 45 min, 1 h, 2 h, 4 h, 6 h, 8 h, 24 h, 48 h and 72 h after dosing.

Blood was withdrawn the v. cephalica antebrachii or v. saphena with sterile, disposable needles.

After sampling, the blood was kept cooled on crushed ice until centrifugation. Plasma samples were prepared by centrifugation of the blood at 2,000 g for 10 minutes at 4° C. within 60 minutes after blood sampling. The separated plasma (approx. 1 ml) was transferred into Eppendorf tubes. Plasma samples were immediately frozen and stored in deep-freezer (−20±5° C.) until analysis.

The concentrations of Telmisartan were determined using a reliable chromatographic bioanalytical method.

### Pharmacokinetic Evaluation

The pharmacokinetic evaluation was performed by using WinNonlin Professional Version 4.0.1 software (Pharsight Corporation, USA). The individual plasma levels versus time curves were evaluated using a non compartmental method.

### Results

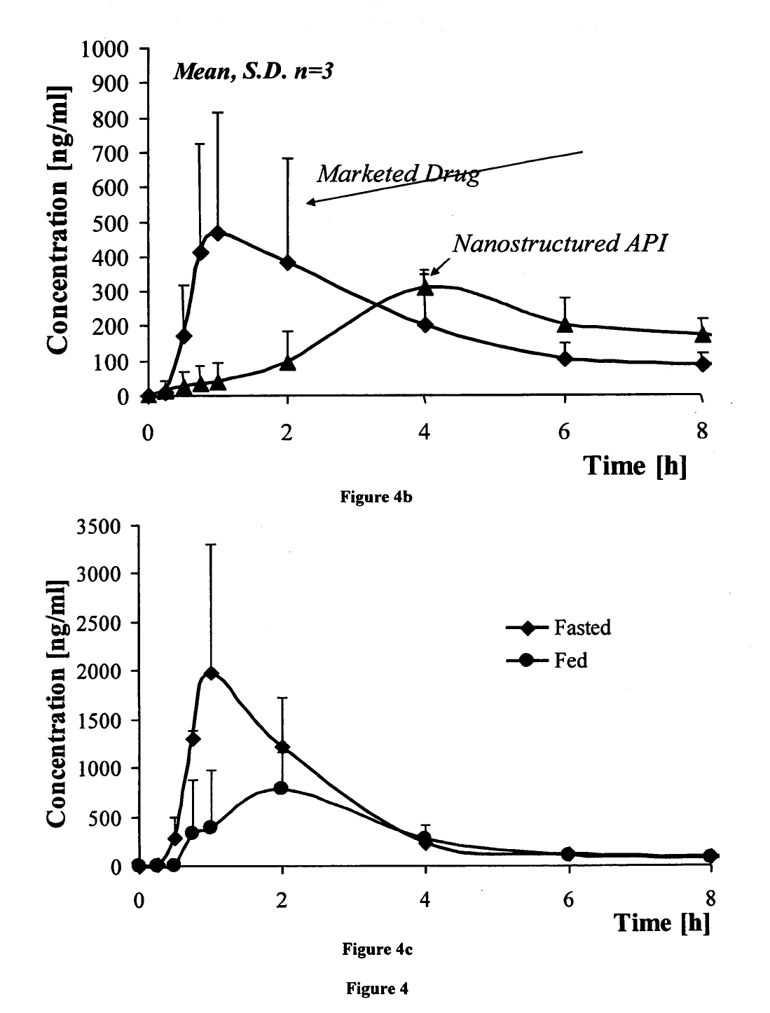

Oral administration of the marketed drug resulted in a fast increase in Telmisartan serum concentrations both in the fasted and in the fed state. The rate of this concentration increase showed very high inter-individual variability. Administration of the nanosized Telmisartan formulation resulted in slower increase in plasma concentrations especially in the fed stated with significantly lower inter-individual differences (FIG. 4.a-b shows plasma concentrations determined in the first 8 hours after oral administration for fasted (a) and fed (b) animals).

Area under the curve for the whole study period (0-72 h) (AUCₗₐₛₜ), cₘₐₓ and tₘₐₓ were determined from the curves and relative bioavailability (Fᵣₑₜ) of the nanosized formulation compared to the marked drug was calculated (table 2). The figures show prolonged tₘₐₓ and reduced Cₘₐₓ for the nanosized formulation, while AUCₗₐₛₜ values were practically identical with relative bioavailability figures 102% and 108% in the fasted and fed states, respectively.

The fast dissolution and absorption parameters of the marketed drug are caused by the unique formulation: Pritor tablets contain solid NaOH. This formulation allows the dissolution of the compound, but it also results in very fast absorption which might not be pharmacologically advantageous. In clinical pharmacology a rapidly occurring, high peak value is not desired, as the temporary high peak concentration might result in side effect. In this case a very rapid fall in blood pressure might cause severe temporary hypotension. The strong alkaline milieu might also modify the absorption of other drugs taken simultaneously. Also, the high inter-individual differences might also be attributed to different degree of alkalinization and consequent differences in the amount of dissolved Telmisartan.

The formulation of example 8 does not contain NaOH, so in order test this hypotheses animal studies were conducted with nanostructured telmisartan and NaOH containing wafer tablets. Higher inter-individual variations were observed when compared to the administration of nanostructured Telmisartan alone. Also, fast increase in plasma concentrations was observed in both the fasted and fed state (FIG. 8.c shows plasma concentrations determined in the first 8 hours after oral administration). Relative bioavailability figures were similar when compared to the marketed drug (97.3% and 133% for fasted and fed conditions, respectively).

Altogether, the nanoformulated API without NaOH added shows bioequivalence to marketed drug tablet without NaOH with a more favorable PK profile.

FIG. 4.: Serum concentrations of Telmisartan after oral administration of 40 mg nanostructured Telmisartan and reference test substance in fasted (a) and fed (b) state. Serum concentrations of Telmisartan after oral administration of nanostructured Telmisartan along with NaOH in the fasted and fed state (c).

Table 2.: Main pharmacokinetic parameters of Telmisartan in female dogs calculated from results presented in FIGS. 4.a and 4.b.

### 2. Dissolution Profiles of the Nanoparticulate Telmisartan Compositions of the Invention

The nanoparticulate Telmisartan compositions of the invention have increased solubility and dissolution profile due to the decreased particles size and unique nanostructured particle formation. Rapid dissolution of an administered active agent is preferable, as faster dissolution generally leads to faster onset of action and greater bioavailability.

### Example 3

### Experimental protocols

Determination of solubility (Cₘₐₓ)

The solubility of nanostructured Telmisartan of example 8 compared to the reference API was determined in distillate water by UV-VIS measurements (Helios Alfa UV spectrophotometer) at 296 nm wavelength and room temperature. The redispersed sample was filtered by 0.20 μm disposable syringe filter. In order to check the nanoparticle presence in the solution, it was irradiated by red laser pointer operating at 670 nm wavelength. If no scattering was observed the filtration was successful, the solution did not contain nanoparticles.

### Determination of Solubility (Cₘₐₓ) in the Presence of Sodium Hydroxide

Pritor tablet contains sodium hydroxide which function is to neutralize the acidic condition and dissolve the Telmisartan during the absorption. To evaluate the effect of sodium hydroxide on solubility, nanostructured Telmisartan was dissolved in the presence of equal amount of sodium hydroxide as Pritor tablet contains.

270.1 mg nanostructured active (40 mg Telmisartan) of example 8, mixture of 270.1 mg of the same nanostructured active (40 mg Telmisartan) and 1.87 mg NaOH and 1 Pritor tablet were dissolved in 100 mL pH=2.5 HCl solution. The suspension was filtered by 0.2 μm disposable syringe filter. In order to check the nanoparticle presence in the solution, it was irradiated by red laser pointer operating at 670 nm wavelength. If no scattering was observed the filtration was successful, the solution did not contain nanoparticles. Telmisartan concentration was determined by UV-VIS measurements (Agilent 8453).

### Dissolution Tests

Dissolution tests were performed by redispersing 5 mg reference Telmisartan and 34.7 mg nanostructured Telmisartan powder containing 5 mg Telmisartan in 10 mL distillate water. The suspension was stirred for 1, 5, 10, 20 and 60 minutes and then it was filtered by 0.2 μm disposable syringe filter. Telmisartan concentration was determined by UV-VIS spectrophotometer (Agilent 8453).

### Results

### Determination of Cₘₐₓ

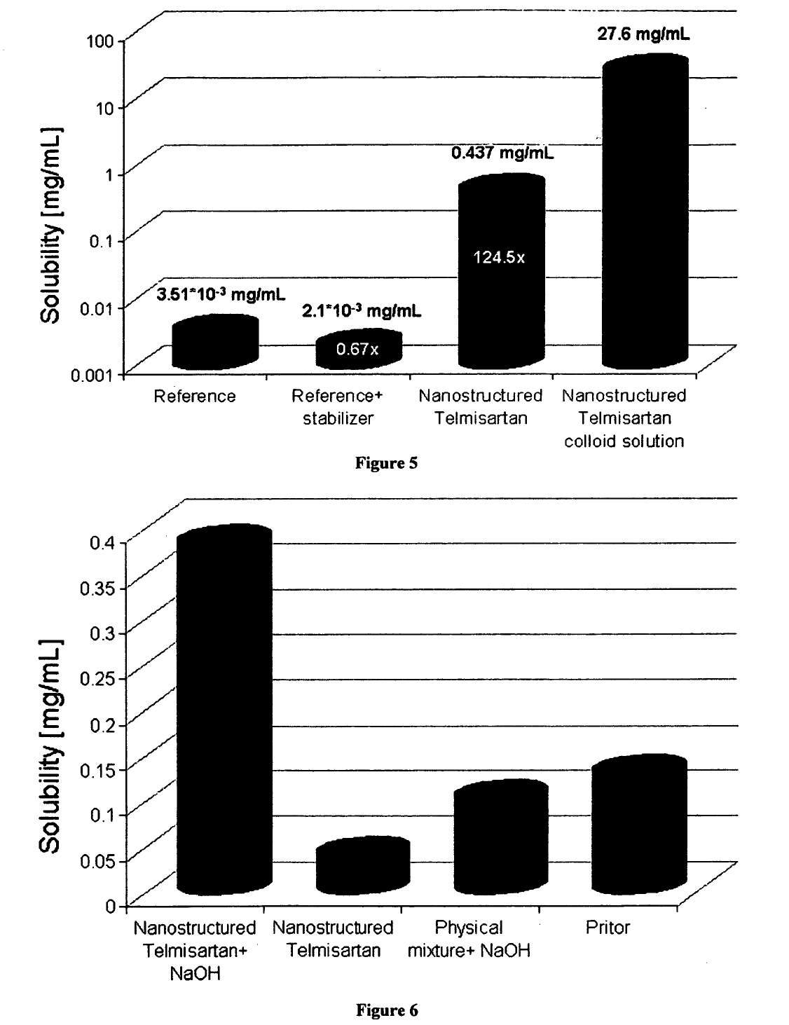

Redispersibility test was performed in order to determine the solubility of the nanostructured Telmisartan. The particle size of the redispersed nanostructured Telmisartan was 104 nm by intensity based average and 26 nm by numeric average. The d(90) values were 185 and 40 nm by intensity based and numeric average, respectively. The solubility of the nanostructured Telmisartan was 0.4 mg/mL which is 124.5 times higher than the solubility of Telmisartan in distillate water (FIG. 5).

FIG. 5.: Solubility enhancement of Telmisartan

### Solubility Test in the Presence of Sodium Hydroxide

Pritor tablet contains sodium hydroxide which function is to neutralize the acidic condition and dissolve the Telmisartan during the absorption. To evaluate the effect of sodium hydroxide on solubility, nanostructured Telmisartan of example 8 was dissolved in the presence of equal amount of sodium hydroxide (46.8 μmol) as Pritor tablet contains. In the presence of sodium hydroxide the solubility of nanostructured Telmisartan in pH=2.5 HCl solution was 2.9 times higher than the solubility of Telmisartan in Pritor tablet (FIG. 6).

FIG. 6.: Solubility enhancement of Telmisartan

### Comparative Dissolution Test

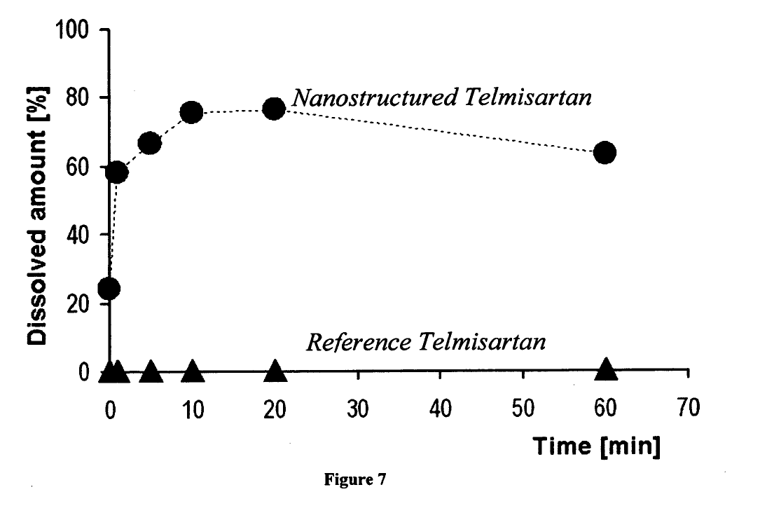

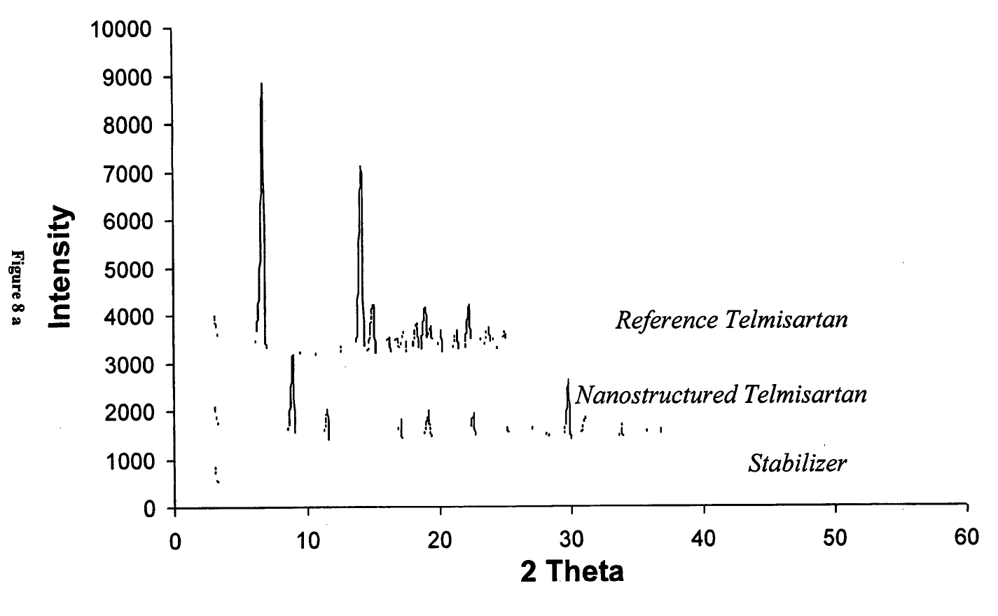

Due to the instantaneous redispersibility of nanostructured Telmisartan of example 8, more than 24% of the Telmisartan content of the composition dissolves immediately upon the redispersion. Within 10 minutes the solution containing the redispersed nanostructured particles reaches its saturated state, the dissolved Telmisartan content is 0.4 mg/mL which is in a good correlation with the solubility of nanostructured Telmisartan (FIG. 7.).

The reference Telmisartan content in distillate water cannot be detected by UV-VIS method.

FIG. 7.: Comparative dissolution test of reference Telmisartan and nanostructured Telmisartan

### 3. Crystallographic Structure of Nanoparticulate Telmisartan Compositions of the Invention

The chemical stability of solid drugs is affected by the crystalline state of the drug. Many drug substances exhibit polymorphism. Each crystalline state has different chemical reactivity. The stability of drugs in their amorphous form is generally lower than that of drugs in their crystalline form, because of the higher free-energy level of the amorphous state.

Decreased chemical stability of solid drugs brought about by mechanical stresses such as grinding is to a change in crystalline state.

The chemical stability of solid drugs is also affected by the crystalline state of the drug through differences in surface area. For reaction that proceeds on the solid surface of drug, an increase in the surface area can increase the amount of drug participating in the reaction.

### Example 4

### Crystallographic Structure Determination

Stable partly crystalline, crystalline, polymorph or amorphous nanostructured Telmisartan compositions of the invention shows significantly enhanced solubility due to its increased surface area when compared to a crystalline reference.

The structure of the Telmisartan nanoparticles prepared by continuous flow nano precipitation method of example 8 was investigated by X-ray diffraction analysis (Philips PW1050/1870 RTG powder-diffractometer). The measurements showed that the nanostructured Telmisartan compositions are partly crystalline or amorphous (See in FIG. 8). The characteristic reflections of the crystalline Telmisartan can be found on the XRD diffractogram of nanosized Telmisartan, but with lower intensity (FIG. 8 a).

FIG. 8: X-ray diffractograms of reference Telmisartan, nanostructured Telmisartan compositions of the invention and stabilizer

### 4. Redispersibility Profiles of the Nanoparticulate Telmisartan Compositions of the Invention

An additional feature of the nanoparticulate Telmisartan compositions of the present invention is that the dried nanoparticles stabilized by surfactant(s)/polymer(s) can be redispersed instantaneously or using traditional redispersants such as mannitol, sucrose.

### Example 5

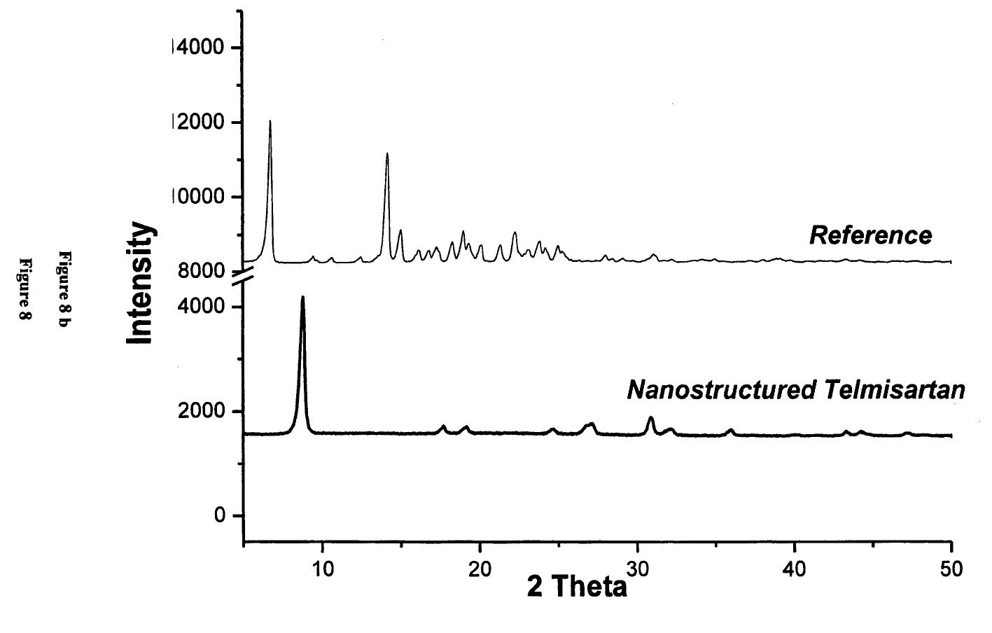

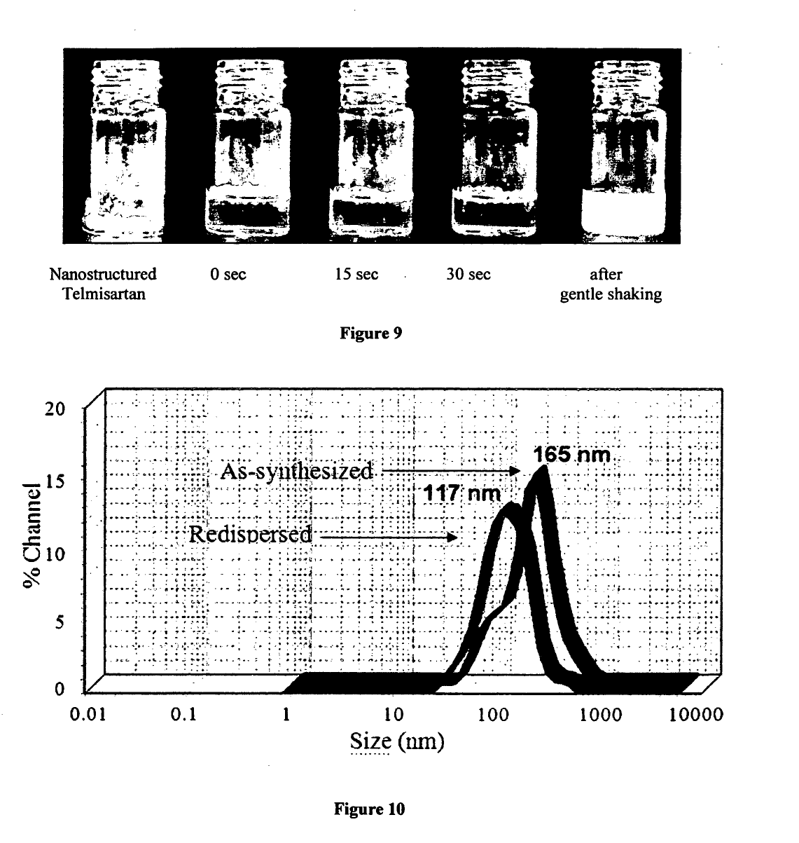

The redispersibility of nanostructured Telmisartan powder of example 8 was performed by dispersing 10 mg nanosized Telmisartan powder in 5 mL distillate water. Following the distillate water addition the vial was gentle shaken by hand resulting colloid dispersion of nanostructured Telmisartan particles as it is demonstrated in FIG. 9. The particle size and size distribution of the redispersed particles can be seen in FIG. 10.

FIG. 9.: Instantaneous redispersibility of nanostructured Telmisartan in distillate water FIG. 10.: Size and size distribution of the Telmisartan nanoparticles before and after the redispersion

### 5. Enhanced Lipophilicity to Increase the Absorption and Permeability Profiles of the Nanoparticulate Telmisartan Compositions of the Invention

Due to the phospholipidic nature of cell membranes, a certain degree of lipophilicity is oftentimes a requirement for the drug compound, not only to be absorbed through the intestinal wall following oral administration but possibly also to exert its pharmacological action in the target tissue. (F. Kesisoglou et al./Advanced Drug Delivery Reviews 59 (2007) 631-644)

The lipophilicity of the Telmisartan can be increased by using lipophilic stabilizer or/and stabilizers having lipophilic side groups on the polymeric backbone and/or amphiphil stabilizers during the nano precipitation. Due to the lipophilic nature or lipophilic side groups of the applied stabilizer, not only the lipophilicity, but the absorption and the permeability of the Telmisartan nanoparticles of the present invention can be increased.

For example using Chitosan, it can increase the paracellular permeability of intestinal epithelia which attributed to the transmucosal absorption enhancement.

Most amphiphilic copolymers employed for drug delivery purposes contain either a polyester or a poly(amino acid)-derivative as the hydrophobic segment. Most of the polyethers of pharmaceutical interest belong to the poloxamer family, i.e. block-copolymers of polypropylene glycol and polyethylene glycol.

### 6. Faster Surface Wetting Profiles of the Nanoparticulate Telmisartan Compositions of the Invention

For the Telmisartan to dissolve, its surface has first to be wetted by the surrounding fluid. The nanosized amorphous/partly crystalline forms possess a chemically randomized surface which expresses hydrophobic and hydrophilic interactions due to the nature of the stabilizer(s) and active pharmaceutical ingredient, which can lead to improved wettability. If the surface of the Telmisartan nanoparticles of the invention is functionalized by hydrophilic groups/stabilizer(s), a higher degree of hydrophility causes faster surface wetting and faster dissolution compared to the original crystalline form. This advanced property of the Telmisartan nanoparticles of the present invention is supported by the results of the redispersibility test. Due to the bigger surface area of the nanostructured Telmisartan particles and the hydrofilic groups of the stabilizer(s) (e.g.: poloxamers, poly(vinylpyrrolidones)) the surface wetting is faster than the reference crystalline form's.

### Example 6

### Visual Observation of Nanoparticulate Telmisartan Wettability

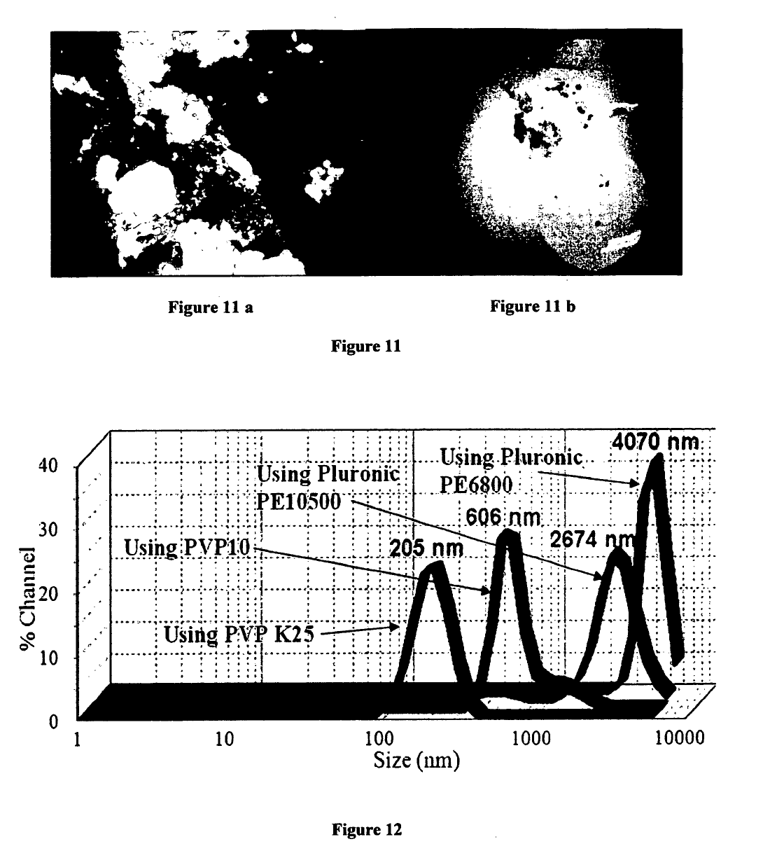

Wettability of nanostructured Telmisartan particles of example 8 was investigated in distillate water and was visualized by stereomicroscope equipped with CCD camera. 0.1 mg reference and nanostructured Telmisartan powder was placed to the slide and then one drop of distillate water was added to the powder. Nanostructured Telmisartan powder started to swell immediately, its wetting was complete, while the reference Telmisartan particles stayed in their aggregated state as it is demonstrated in FIG. 11.

FIG. 11.: Wettability of reference Telmisartan (a) and nanostructured Telmisartan (b) observed by stereomicroscope in 100× magnification

### B. Compositions

The invention provides nanosized Telmisartan nanostructured particle formations comprising at least one stabilizer to stabilize them sterically and/or electrostatically.

The stabilizers preferably are associated or interacted with the Telmisartan, but do not chemically react with the Telmisartan or themselves.

The nanoparticles of Telmisartan of the invention can be formed by solvent-antisolvent precipitation methods using stabilizer(s). The stability of the prepared colloid solution of nanosized Telmisartan can be increased by the combination of additional stabilizer(s) which can act as a second steric or electrostatic stabilizer. Moreover, using additional stabilizer the particle size of Telmisartan of the invention can be decreased and controlled.

### Particle Size of Telmisartan Nanoparticles

The invention contains Telmisartan nanoparticles, which have an average particle size of less than about 600 nm as measured by dynamic light scattering method.

By “an average particle size of less than about 600 nm” it is meant that at least 90% of the Telmisartan nanoparticles have a particle size of less than the average, by number/intensity, i.e., less than about 600 nm, etc., when measured by the above-noted technique.

### Example 7

### Nanostructured Telmisartan Production

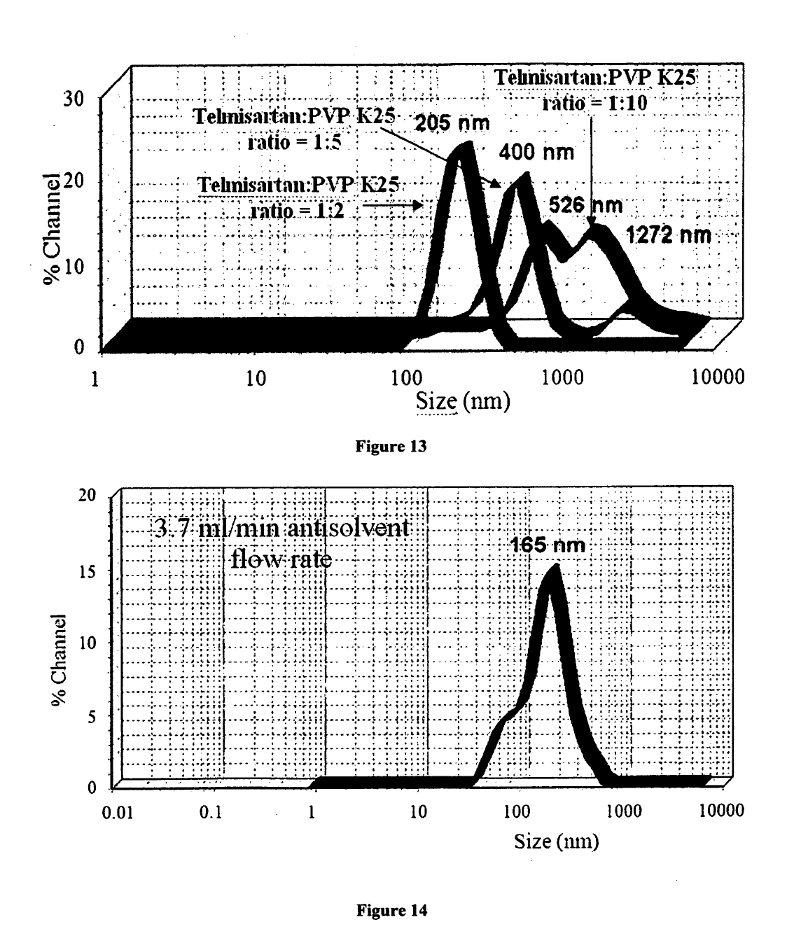

During the experiments Telmisartan nanoparticles were prepared in a microfluidic based continuous flow reactor. As a starting solution, 100 mg Telmisartan, 20 mg sodium dodecyl sulfate and 200 mg poly(vinylpyrrolidone), PVP K-25 dissolved in 100 mL DMSO was used. The prepared solution was passed into the reactor unit with 0.5 mL/min flow rate using a feeding unit. Meanwhile, using a second feeding unit, distilled water was passed into a mixing unit with 2 mL/min flow rate, where it was mixed with the solution containing Telmisartan coming from the first reactor unit. The nanoparticles are continuously produced at atmospheric pressure due to the chemical precipitation by water passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates; pressure and the types of the stabilizers (see FIG. 12). The particles size and size distribution of the Telmisartan particles can be controlled by the amount the stabilizer (PVP K-25) as it is show in FIG. 13. The particles size of the Telmisartan particle was 205 nm in the best case.

FIG. 12.: Particle size and size distribution of Telmisartan nanoparticles using different stabilizers

FIG. 13.: Effect of the stabilizer concentration on the particle size and size distribution of Telmisartan nanoparticles

### Example 8

### Nanostructured Telmisartan Production

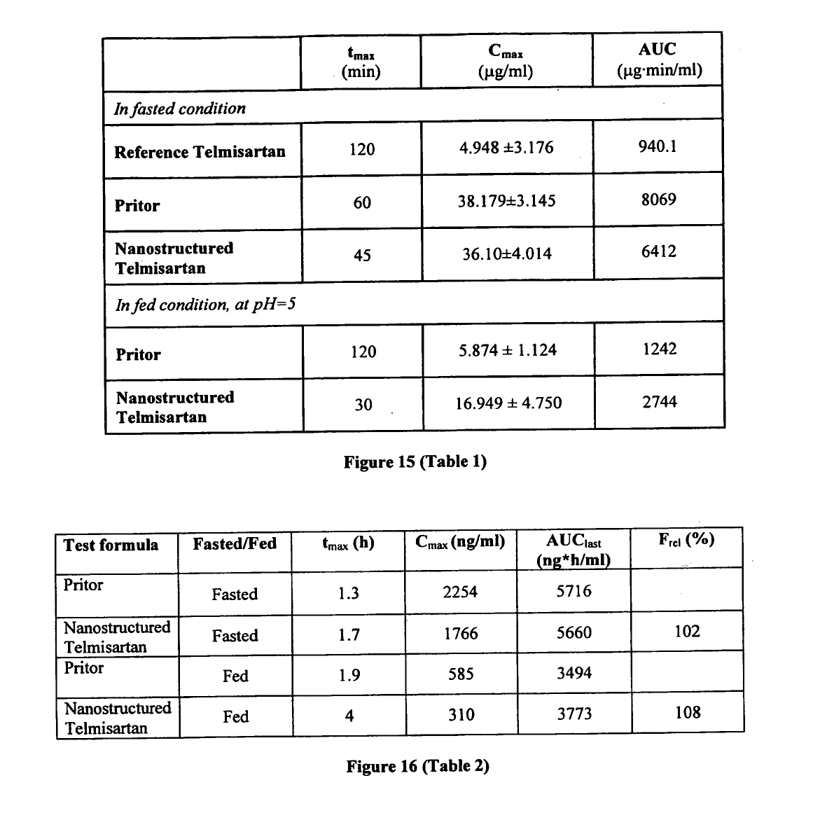

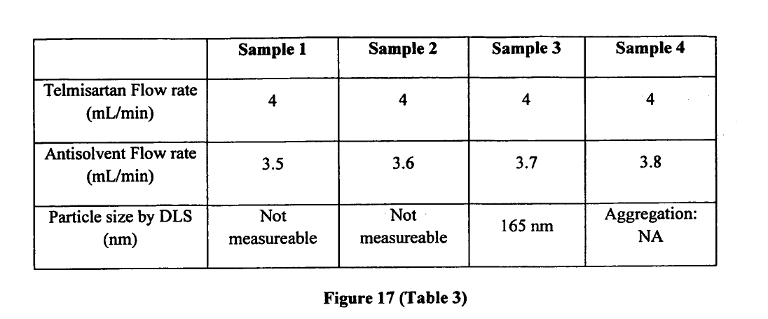

During the experiments Telmisartan nanoparticles were prepared in a microfluidic based continuous flow reactor. As a starting solution, 160 mg Telmisartan and 320 mg poly(vinylpyrrolidone), PVP40 dissolved in 80 mL 0.1 M NaOH solution was used. The prepared solution was passed into the reactor unit with 4 mL/min flow rate using a feeding unit. Meanwhile, using a second feeding unit, 0.1 M acetic acid solution was passed into a mixing unit with 3.7 mL/min flow rate, where it was mixed with the solution containing Telmisartan coming from the first reactor unit. The nanoparticles are continuously produced at atmospheric pressure due to the chemical precipitation by acetic acid passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates. The particles size of the Telmisartan particle was 165 nm in the best case as shown in FIG. 14 and Table 3.

FIG. 14.: Particle size and size distribution of Telmisartan nanoparticles

Table 3.: Effect on the flow rates on the particle size of Telmisartan

### Example 9

### Telmisartan Nanoparticles Loaded Cream Formulation

Preparing 100 mL gel containing Telmisartan nanoparticles 1.3 g Carbopol 971 was dissolved under vigorous stirring at room temperature in 100 mL Telmisartan colloidal solution as synthesized by the method described in example 8.

1-3. (canceled)

4. A stable nanostructured Telmisartan composition comprising:
(a) nanostructured Telmisartan having an average particle size of less than about 600 nm; and
(b) at least one stabilizer,
wherein the composition is prepared in a continuous flow reactor.

5. The stable nanostructured Telmisartan composition according to claim 4, comprising:
(a) nanostructured Telmisartan having an average particle size of less than about 600 nm; and
(b) at least one stabilizer,
wherein the composition is prepared in a microfluidic based continuous flow reactor.

6. The composition according to claim 4, wherein the average particle size is between 600 nm and 50 nm.

7. (canceled)

8. The composition according to claim 4, wherein the average particle size is between 200 nm and 50 nm.

9. The composition of claim 4 wherein the stabilizer is selected from the group consisting of hydroxypropyl methylcellulose, hydroxypropylcellulose, poly(vinylpyrrolidone), sodium dodecyl sulfate, sodium lauryl sulfate, gelatin, dextran, stearic acid, glycerol monostearate, cetostearyl alcohol, sorbitan esters, polyoxyethylene castor oil derivatives, poly(meth)acrylate-based polymers and copolymers, acetic acid ethenyl ester polymer with 1-ethenyl-2-pyrrolidinone (PVP/VA copolymers), sodium dodecyl benzene sulfonate, tocopheryl polyethylene glycol succinates, polyethoxylated castor oils and its derivatives, polyoxyethylene sorbitan fatty acid esters; polyethylene glycols, polyoxyethylene stearates, methylcellulose, hydroxy ethylcellulose, cellulose acetate phthalate, polyvinyl alcohol, 4-(1,1,3,3-tetramethylbutyl)-phenol polymer with ethylene oxide and formaldehyde, poloxamers; poloxamines, which is a tetrafunctional block copolymer derived from sequential addition of propylene oxide and ethylene oxide to ethylenediamine; PEG-phospholipid, PEG-cholesterol, PEG-cholesterol derivative, PEG-vitamin A, PEG-vitamin E, lysozyme, poly(2-ethyl-2-oxazoline), poly(methyl vinyl ether), random copolymers of vinyl pyrrolidone and vinyl acetate.

10. The composition of claim 9, where the composition contains additional stabilizer which is hydroxyl-propyl-cellulose derivatives, or combination of two stabilizers of claim 9 and/or lauryl trimethyl ammonium chloride, alkylbenzyl methyl ammonium chloride, alkylbenzyl dimethyl ammonium bromide, benzyl trimethylammonium bromide.

11. A process for the preparation of nanostructured Telmisartan composition according to claim 4, comprising precipitating nanostructured Telmisartan from a solution of Telmisartan and at least one stabilizer, if desired in the presence of a pharmaceutically acceptable acid, in a continuous flow reactor.

12. The process of claim 11 using as a continuous flow reactor a microfluidic based continuous flow reactor.

13. (canceled)

14. (canceled)

15. The process of claim 11, comprising (1) dissolving Telmisartan and one or more stabilizers in an alkali-hydroxide solution; (2) adding to the solution of step (1) a solution of a pharmaceutically acceptable acid comprising optionally one or more stabilizers and (3) precipitating the formulation from step (2).

16. (canceled)

17. Process according to claim 15, wherein the alkali-hydroxide solution is sodium-hydroxide solution.

18. The process of claim 15, comprising the use of two different solvents miscible with each other, where Telmisartan is soluble only in one of them.

19. A pharmaceutical composition comprising a nanostructured composition according to claim 4 together with pharmaceutically acceptable auxiliary materials.

20. The pharmaceutical composition according to claim 19, wherein the composition is free of sodium-hydroxide content.

21-22. (canceled)

23. Use of nanostructured Telmisartan of claim 4 for preparation of a medicament.

24. Use of the nanostructured composition according to claim 4 or a pharmaceutical composition comprising the nanostructured composition according to 4 together with pharmaceutically acceptable auxiliary materials for the treatment hypertension.

25-29. (canceled)

30. The use according to claim 24, wherein the composition has
a solubility at least about 0.4 mg/ml in water,
instantaneous redispersibility in physiological mediums,
reduced food and side effect,
absorption in human gastrointestinal tract,
faster onset of action,
for decreasing the dosage used.

31. A method of treating a subject in need by administering to the subject an effective amount of the nanostructured Telmisartan of claim 4 or a pharmaceutical composition comprising the nanostructured composition according to 4 together with pharmaceutically acceptable auxiliary materials.

32. The method of claim 31, wherein the composition has
a solubility at least about 0.4 mg/ml in water,
instantaneous redispersibility in physiological mediums,
reduced food and side effect,
absorption in human gastrointestinal tract,
faster onset of action,
for decreasing the dosage used.

33. The method of claim 31, wherein the subject suffers from hypertension.
